(19) United States
(12) Patent Application Publication
(10) Pub. No.: US 2011/0177163 A1
(43) Pub. Date: Jul. 21, 2011

(54) COMPOSITIONS AND METHODS FOR TREATING HEPATITIS A

(75) Inventors: Francisco Diaz-Mitoma, Ottawa (CA); Thanh Le, Ottawa (CA); David E. Anderson, Boston, MA (US)

(73) Assignee: VARIATION BIOTECHNOLOGIES, INC., Gatineau, QC (CA)

(21) Appl. No.: 13/119,583

(22) PCT Filed: Sep. 18, 2009
(86) PCT No.: PCT/US09/57492

§ 371 Date: Mar. 17, 2011

Related U.S. Application Data
(60) Provisional application No. 61/098,177, filed on Sep. 18, 2008.

(51) Int. Cl. A61K 39/29; A61K 9/50; A61K 9/00; A61P 31/12

(52) U.S. Cl. 424/451; 424/226.1; 424/400

(57) ABSTRACT

The present application provides compositions and methods useful for treating hepatitis A. In particular, while hepatitis A vaccines are currently limited to parenteral administration routes (i.e., intramuscular injection), we have identified compositions that induce a protective response when administered orally.

### RELATED APPLICATIONS

This application claims priority to U.S. Provisional Application No. 61/098,177 filed Sep. 18, 2009, the content of which is hereby incorporated by reference in its entirety.

### BACKGROUND

Hepatitis A is a serious liver disease caused by the hepatitis A virus (HAV). The virus is found in the stools of persons with hepatitis A. HAV is transmitted from person to person, primarily by the fecal-oral route. The incidence of hepatitis A is closely related to socioeconomic development, and sero-epidemiological studies show that prevalence of anti-HAV antibodies in the general population varies from 15% to close to 100% in different parts of the world. An estimated 1.5 million clinical cases of hepatitis A occur each year (e.g., see Centers for Disease Control and Prevention. Prevention of hepatitis A through active or passive immunization. Recommendation of the Advisory Committee on Immunization Practices (ACIP). Morbidity and Mortality Weekly Report, 1999, 48(RR-12):1-37).

Several inactivated hepatitis A vaccines are currently licensed. For example, HAVRIX® is developed and manufactured by GlaxoSmithKline Biologicals. HAVRIX contains a sterile suspension of formalin inactivated HAV. The viral antigen activity is referenced to a standard using an ELISA and expressed in terms of ELISA Units (U). Each 1 ml adult dose of vaccine consists of 1440 U of viral antigen, adsorbed on 0.5 mg of aluminum as aluminum hydroxide (alum). HAVRIX (as with all other licensed hepatitis A vaccines) is supplied as a sterile suspension for intramuscular (IM) administration. Although one dose of HAVRIX provides at least short-term protection, a second booster dose after six to twelve months is currently recommended to ensure long-term protection.

Another example of an inactivated hepatitis A vaccine, AIMMUGEN® has been licensed and marketed in Japan since 1994 by Kaketsuken. AIMMUGEN contains a sterile suspension of formaldehyde inactivated HAV. The recommended adult dose is 0.5 μg IM at 0, 1 and 6 months.

While HAVRIX, AIMMUGEN and other licensed hepatitis A vaccines have been successful in reducing the incidence of hepatitis A worldwide there remains a need in the art for an orally delivered hepatitis A vaccine.

### SUMMARY

The present application provides compositions and methods useful for treating hepatitis A. In particular, while hepatitis A vaccines are currently limited to parenteral administration routes (i.e., intramuscular injection), we have identified compositions that induce a protective response when administered orally.

### BRIEF DESCRIPTION OF THE DRAWINGS

FIG. 1 is a graph which shows vesicle size data that was obtained by analyzing exemplary vesicles by dynamic light scattering on a Malvern Instrument MASTERSIZER™ 2000.

FIG. 2 shows the systemic (serum samples) IgG responses against hepatitis A viral antigen that were obtained after oral vaccination of monkeys with an exemplary immunogenic composition. Three rhesus macaques were immunized by gastric gavage on days 0, 14 and 28 with an exemplary immunogenic composition (inactivated Hepatitis A antigen, HAVRIX equivalent to 4320 U/dose, with poly (I:C) in a vesicle formulation). Serum samples collected 14 days and 28 days after the last immunization were tested by ELISA against inactivated hepatitis A viral antigen.

FIG. 3 shows the mucosal (nasal wash samples) IgA responses against hepatitis A viral antigen that were obtained after oral vaccination of monkeys with an exemplary immunogenic composition. Three rhesus macaques were immunized by gastric gavage on days 0, 14 and 28 with an exemplary immunogenic composition (inactivated hepatitis A antigen, HAVRIX equivalent to 4320 U/dose, with poly (I:C) in a vesicle formulation). Nasal wash samples collected 14 days after the second vaccination (P2Vd14), and 14 and 28 days after the third vaccination (P3Vd14 and P3Vd28, respectively) were tested by ELISA against inactivated hepatitis A viral antigen.

FIG. 4 shows the systemic (serum samples) IgG responses against hepatitis A viral antigen that were obtained after oral vaccination of BALB/c mice with an exemplary immunogenic composition. Mice were immunized by oral gavage on days 0, 3, 14 and 17 with an exemplary immunogenic composition (inactivated hepatitis A antigen from Meridian Life Sciences, Meridian HAV-ag equivalent to 2 μg antigen, with or without poly (I:C) in a vesicle formulation). Serum samples collected prior to study start and on days 29, 44, 71, 100, 129, 164 and 191 after the first immunization were tested by ELISA against inactivated hepatitis A viral antigen.

### DEFINITIONS

Throughout the present application, several terms are employed that are defined in the following paragraphs.

As used herein, the term “immune response” refers to a response elicited in an animal. An immune response may refer to cellular immunity, humoral immunity or may involve both. An immune response may also be limited to a part of the immune system. For example, in certain embodiments, an immunogenic composition may induce an increased IFNγ response. In certain embodiments, an immunogenic composition may induce a mucosal IgA response (e.g., as measured in nasal and/or rectal washes). In certain embodiments, an immunogenic composition may induce a systemic IgG response (e.g., as measured in serum).

As used herein, the term “immunogenic” means capable of producing an immune response in a host animal against a non-host entity (e.g., a hepatitis A virus). In certain embodiments, this immune response forms the basis of the protective immunity elicited by a vaccine against a specific infectious organism (e.g., a hepatitis A virus).

As used herein, the terms “therapeutically effective amount” refer to the amount sufficient to show a meaningful benefit in a subject being treated. The therapeutically effective amount of an immunogenic composition may vary depending on such factors as the desired biological endpoint, the nature of the composition, the route of administration, the health, size and/or age of the subject being treated, etc.

As used herein, the term “treat” (or “treating”, “treated”, “treatment”, etc.) refers to the administration of an immunogenic composition to a subject who has hepatitis A, a symptom of hepatitis A or a predisposition toward hepatitis A, with the purpose to alleviate, relieve, alter, ameliorate, improve or affect the hepatitis A infection, a symptom or symptoms of hepatitis A, or the predisposition toward hepatitis A. In certain embodiments, the term “treating” refers to the vaccination of a subject.

As used herein the expression “HAV antigen” refers to any antigen capable of stimulating neutralizing antibody to HAV in humans. The HAV antigen may comprise inactivated virus particles or live attenuated virus particles.

### DETAILED DESCRIPTION OF CERTAIN EMBODIMENTS

The present application provides immunogenic compositions and methods of using these to treat hepatitis A. Of particular note, while hepatitis A vaccines are currently limited to parenteral administration routes (i.e., intramuscular injection), we have identified immunogenic compositions that induce a protective response when administered orally.

In general, an immunogenic composition of the present disclosure includes an inactivated or attenuated hepatitis A virus in combination with a vesicle. The vesicle includes certain components that are described in more detail herein. In certain embodiments, an immunogenic composition may also include an adjuvant.

### I. Inactivated or Attenuated Hepatitis A Virus

In one aspect, the present application provides immunogenic compositions that include an inactivated or attenuated hepatitis A virus (also called “hepatitis A viral antigen”, “HAV antigen” or “viral antigen” herein).

As mentioned above, all known hepatitis A vaccines include an inactivated hepatitis A virus. It is to be understood that any one of these licensed hepatitis A vaccines may be combined with a vesicle as described herein to produce an immunogenic composition. Indeed, among other things we have demonstrated that commercial HAVRIX may be combined in this manner to produce an orally active immunogenic composition without any purification (e.g., to remove alum adjuvant or other reagents in the vaccine). This outcome was surprising since HAVRIX and other inactivated hepatitis A viral antigens have previously only been shown to be immunogenic when administered parenterally. The benefits of combining an inactivated viral antigen (as opposed to a subunit viral antigen) with the vesicles of the present invention were also unexpected.

It will be appreciated that any method may be used to prepare an inactivated hepatitis A virus. In general, these methods will involve propagating a hepatitis A virus in a host cell, lysing the host cell to release the virus, isolating and then inactivating the viral antigen. For example, in preparing HAVRIX, hepatitis A virus strain HM175 is propagated in MRC-5 human diploid cells. After removal of the cell culture medium, the cells are lysed to form a suspension. This suspension is purified through ultrafiltration and gel permeation chromatography procedures. The purified lysate is then treated with formalin to ensure viral inactivation (e.g., see Andre et al., Prog. Med. Virol. 37:72-95, 1990).

In preparing AIMMUGEN, hepatitis A virus strain KRM0003 (established from a wild-type HAV, which had been isolated from the feces of a hepatitis A patient) is propagated in GL37 cells (a cell strain established for vaccine production from a parent cell strain of African green monkey kidney). The GL37 cells are inoculated with HAV strain KRM0003 and viral antigen is harvested, extensively purified and inactivated with formaldehyde.

Another example of an inactivated hepatitis A virus that is commercially available but is not a licensed vaccine is hepatitis A antigen (HAV-ag) from Meridian Life Sciences. Like HAVRIX the Meridian HAV-ag also derives from hepatitis A virus strain HM175 but it is propagated in FRhK-4 (fetal rhesus kidney) cells. After removal of cell culture medium, the cells are lysed to form a suspension and the suspension is partially purified by gradient centrifugation and inactivated by treatment with formalin.

It will be appreciated that any hepatitis A virus strain may be used, e.g., without limitation any of the following strains which have been described in the art (and other non-human variants):
Human hepatitis A virus Hu/Arizona/HAS-15/1979
Human hepatitis A virus Hu/Australia/HM175/1976
Human hepatitis A virus Hu/China/H2/1982
Human hepatitis A virus Hu/Costa Rica/CR326/1960
Human hepatitis A virus Hu/France/CF-53/1979
Human hepatitis A virus Hu/Georgia/GA76/1976
Human hepatitis A virus Hu/Germany/GBM/1976
Human hepatitis A virus Hu/Japan/HAJ85-1/1985
Human hepatitis A virus Hu/Los Angelos/LA/1975
Human hepatitis A virus Hu/Northern Africa/MBB/1978
Human hepatitis A virus Hu/Norway/NOR-21/1998
Human hepatitis A virus Hu/Sierra Leone/SLF88/1988
Human hepatitis A virus MSM1
Human hepatitis A virus Shanghai/LCDC-1/1984

In addition, while formalin and formaldehyde are commonly used to inactivate licensed hepatitis A vaccines it is to be understood that other techniques could be used, e.g., treatment with chlorine, exposure to high temperatures (the viral antigen is inactivated above 85° C./185° F.), etc.

In certain embodiments it may prove advantageous to add additional steps to the traditional method for preparing an inactivated hepatitis A virus. For example, U.S. Pat. No. 6,991,929 describes including a protease treatment step (e.g., trypsin) after the virus has been propagated. This step was found to improve the removal of host cell material and yield a purer viral preparation.

While all currently licensed hepatitis A vaccines include inactivated viral antigens, alternative vaccines which include attenuated viral antigen have also been described in the literature. In certain embodiments, an immunogenic composition may comprise such an attenuated viral antigen. As is well known in the art, the advantage of an attenuated vaccine lies in the potential for higher immunogenicity which results from its ability to replicate in vivo without causing a full infection.

One method which has been used in the art to prepare attenuated hepatitis A viruses is viral adaptation which involves serially passing a viral strain through multiple cell cultures. Over time the strain mutates and attenuated strains can then be identified. In certain embodiments the virus may be passed through different cell cultures. For example, researchers have generated attenuated hepatitis A viruses by passing strain CR326 sixteen times in human diploid lung (MRCS) cell cultures (see Provost et al., J. Med. Virol. 20:165-175, 2005). A slightly more virulent strain was obtained by passing the same strain fifteen times in fetal rhesus monkey kidney (FRhK6) cell cultures plus eight times in MRCS cell cultures. An alternative attenuated hepatitis A vaccine which was prepared in this fashion from the H2 strain has also been described (see European Patent No. 0413637 and Mao et al., Vaccine 15:944-947, 1997).

In certain embodiments it may prove advantageous to perform one or more of the cell culture steps at a reduced temperature. For example, European Patent No. 0413637 describes including one or more inoculation steps in which the temperature is reduced (e.g., to 32-34° C. instead of 35-36° C.).

U.S. Pat. No. 6,180,110 describes an attenuated hepatitis A virus (HAV 4380) which grows in MRC-5 cells. The researchers identified mutations in HAV 4380 which appeared to be associated with attenuation by comparing its genome with the genome of a more virulent strain. This allowed them to design mutant HAV strains with optimal characteristics for a candidate attenuated hepatitis A vaccine. It will be appreciated that this approach could be applied to any known attenuated hepatitis A virus and used to genetically engineer variants without the need for viral adaptation.

### II. Vesicles

As mentioned above, immunogenic compositions of the present disclosure include a vesicle. As is well known in the art, vesicles generally have an aqueous compartment enclosed by one or more bilayers which include amphipathic molecules (e.g., fatty acids, lipids, steroids, etc.). In certain embodiments, the components of the viral antigen will be present in the aqueous core of the vesicle. However, depending on their hydrophobicity, components of the viral antigen may also be associated with a bilayer (e.g., through hydrophobic interactions and/or hydrogen or ionic bonds). In certain embodiments an immunogenic composition may also include amounts or components of the viral antigen that are not entrapped within a vesicle.

In general, the vesicles of the present disclosure comprise a non-ionic surfactant and a transport enhancing molecule which facilitates the transport of lipid-like molecules across mucosal membranes.

### Non-Ionic Surfactant

Any non-ionic surfactant with appropriate amphipathic properties may be used to form a vesicle. Without limitation, examples of suitable surfactants include ester-linked surfactants based on glycerol. Such glycerol esters may comprise one of two higher aliphatic acyl groups, e.g., containing at least ten carbon atoms in each acyl moiety. Surfactants based on such glycerol esters may comprise more than one glycerol unit, e.g., up to 5 glycerol units. Glycerol monoesters may be used, e.g., those containing a C₁₂-C₂₀alkanoyl or alkenoyl moiety, for example caproyl, lauroyl, myristoyl, palmitoyl, oleyl or stearoyl. An exemplary surfactant is 1-monopalmitoyl glycerol.

Ether-linked surfactants may also be used as the non-ionic surfactant. For example, ether-linked surfactants based on glycerol or a glycol having a lower aliphatic glycol of up to 4 carbon atoms, such as ethylene glycol, are suitable. Surfactants based on such glycols may comprise more than one glycol unit, e.g., up to 5 glycol units (e.g., diglycolcetyl ether and/or polyoxyethylene-3-lauryl ether). Glycol or glycerol monoethers may be used, including those containing a C₁₂-C₂₀alkanyl or alkenyl moiety, for example capryl, lauryl, myristyl, cetyl, oleyl or stearyl. Ethylene oxide condensation products that can be used include those disclosed in PCT Publication No. WO88/06882 (e.g., polyoxyethylene higher aliphatic ether and amine surfactants). Exemplary ether-linked surfactants include 1-monocetyl glycerol ether and diglycolcetyl ether.

### Transport Enhancing Molecule

The vesicles also comprise a transport enhancing molecule which facilitates the transport of lipid-like molecules across mucosal membranes. As described in U.S. Pat. No. 5,876,721, a variety of molecules may be used as transport enhancers. For example, cholesterol derivatives in which the C₂₃ carbon atom of the side chain carries a carboxylic acid, and/or derivatives thereof, may be used as transport enhancers. Such derivatives include, but are not limited to, the “bile acids” cholic acid and chenodeoxycholic acid, their conjugation products with glycine or taurine such as glycocholic and taurocholic acid, derivatives including deoxycholic and ursodeoxycholic acid, and salts of each of these acids.

Other transport enhancers include acyloxylated amino acids, such as acylcarnitines and salts thereof. For example, acylcarnitine containing C₆₋₂₀alkanoyl or alkenoyl moieties, such as palmitoylcarnitine, may be used as transport enhancers. As used herein, the term acyloxylated amino acid is intended to cover primary, secondary and tertiary amino acids as well as α, β, and γ amino acids. Acylcarnitines are examples of acyloxylated γ amino acids.

It is to be understood that vesicles may comprise more than one type of transport enhancer, e.g., one or more different bile salts and one or more acylcarnitines.

### Ionic Amphiphile

In certain embodiments, the vesicles may also incorporate an ionic amphiphile, e.g., to cause the vesicles to take on a negative charge. For example, this may help to stabilize the vesicle and provide effective dispersion. Without limitation, acidic materials such as higher alkanoic and alkenoic acids (e.g., palmitic acid, oleic acid) or other compounds containing acidic groups including phosphates such as dialkyl phosphates (e.g., dicetylphospate, or phosphatidic acid or phosphatidyl serine) and sulphate monoesters such as higher alkyl sulphates (e.g., cetylsulphate), may all be used for this purpose.

### Methods for Preparing Vesicles

In certain embodiments, vesicles may be formed by admixing the non-ionic surfactant and transport enhancer with an appropriate hydrophobic material of higher molecular mass capable of forming a bi-layer (such as a steroid, e.g., a sterol such as cholesterol). The presence of the steroid may assist in forming the bi-layer on which the physical properties of the vesicle depend.

It will be appreciated however that vesicles may be made by modifications of any known technique for preparing vesicles comprising non-ionic surfactants, such as those described in PCT Publication No. WO1993/019781. An exemplary technique is the rotary film evaporation method, in which a film of non-ionic surfactant is prepared by rotary evaporation from an organic solvent, e.g., a hydrocarbon or chlorinated hydrocarbon solvent such as chloroform, e.g., see Russell and Alexander, J. Immunol. 140:1274 (1988). The resulting thin film is then rehydrated in bicarbonate buffer in the presence of the transport enhancer.

Another method for the production of vesicles may be based on a method that is disclosed by Collins et al., J. Pharm. Pharmacol. 42:53 (1990). This method involves melting a mixture of the non-ionic surfactant, steroid (if used) and ionic amphiphile (if used) and hydrating with vigorous mixing in the presence of aqueous buffer. The transport enhancer can be incorporated into the vesicles, either by being included with the other constituents in the melted mixture or concomitantly during the process used to entrap the viral antigen (see below).

Another method involves hydration in the presence of shearing forces. An apparatus that can be used to apply such shearing forces is well known, suitable equipment (see, e.g., PCT Publication No. WO88/06882). Sonication and ultra-sonication are also effective means to form the vesicles or to alter their particle size.

### Methods for Entrapping Viral Antigen

The viral antigen may be associated with vesicles in any manner. For example, in the rotary film evaporation technique, this can be achieved by hydration of the film in the presence of viral antigen together with the transport enhancer. In other methods, the viral antigen may be associated with preformed vesicles by a dehydration-rehydration method in which viral antigen present in the aqueous phase is entrapped by flash freezing followed by lyophilisation, e.g., see Kirby and Gregoriadis, Biotechnology 2:979 (1984). Alternatively a freeze thaw technique may be used in which vesicles are mixed with viral antigen and repeatedly flash frozen in liquid nitrogen, and warmed to a temperature of the order of, e.g., 60° C. (i.e., above the transition temperature of the relevant surfactant), e.g., see Pick, Arch. Biochem. Biophys. 212:195 (1981). In addition to entrapping viral antigen, the dehydration-rehydration method is also capable of concomitantly incorporating additional transport enhancers into the vesicles.

In each of these methods, the suspension of vesicle components may be extruded several times through microporous polycarbonate membranes at an elevated temperature sufficient to maintain the vesicle-forming mixture in a molten condition. This has the advantage that vesicles having a uniform size may be produced. It will be appreciated that a composition will typically include a mixture of vesicles with a range of sizes. It is to be understood that the diameter values listed below correspond to the most frequent diameter within the mixture. In various embodiments >90% of the vesicles in a composition will have a diameter which lies within 50% of the most frequent value (e.g., 1000±500 nm). In certain embodiments the distribution may be narrower, e.g., >90% of the vesicles in a composition may have a diameter which lies within 40, 30, 20, 10 or 5% of the most frequent value.

In general, vesicles that may be used in accordance with the invention may be of any size. In certain embodiments, the composition may include a vesicle with a diameter in the range of about 150 nm to about 15 μm, e.g., about 800 nm to about 1.5 μm. In certain embodiments, the vesicle may have a diameter which is greater than 10 μm, e.g., about 15 μm to about 25 μm. In certain embodiments, the vesicle may have a diameter in the range of about 2 μm to about 10 μm, e.g., about 1 μm to about 4 μm. In certain embodiments, the vesicle may have a diameter which is less than 150 nm, e.g., about 50 nm to about 100 nm.

The transport enhancer(s) will generally be present in amount of between 15 and 100% percent by weight of the non-ionic surfactant. In some embodiments, the transport enhancer(s) comprise between 20 and 50% by weight of the non-ionic surfactant. A steroid, if present, will typically comprise between 20 and 120% by weight of the non-ionic surfactant, e.g., between 40 and 100% or 50 and 80%. An ionic amphiphile, if present, will typically comprise, between 10 and 50% by weight of the non-ionic surfactant, e.g., between 15 and 40% or 20 and 30%.

### III. Adjuvants

In certain embodiments, immunogenic compositions may include one or more adjuvants. As is well known in the art, adjuvants are agents that enhance immune responses. Adjuvants are well known in the art (e.g., see “Vaccine Design: The Subunit and Adjuvant Approach”, Pharmaceutical Biotechnology, Volume 6, Eds. Powell and Newman, Plenum Press, New York and London, 1995).

Exemplary adjuvants include complete Freund's adjuvant (CFA), incomplete Freund's adjuvant (IFA), squalene, squalane and alum (aluminum hydroxide), which are materials well known in the art, and are available commercially from several sources. In certain embodiments, aluminum or calcium salts (e.g., hydroxide or phosphate salts) may be used as adjuvants. Alum (aluminum hydroxide) has been used in many existing vaccines. Typically, about 40 to about 700 μg of aluminum is included per dose when given IM. For example, HAVRIX includes 500 μg of aluminum per dose. Exemplary immunogenic compositions that are described in the examples were prepared using unpurified commercial HAVRIX and therefore included alum as an adjuvant.

In various embodiments, oil-in-water emulsions or water-in-oil emulsions can also be used as adjuvants. For example, the oil phase may include squalene or squalane and a surfactant. In various embodiments, non-ionic surfactants such as the mono- and di-C₁₂-C₂₄-fatty acid esters of sorbitan and mannide may be used. The oil phase preferably comprises about 0.2 to about 15% by weight of the immunogenic composition (e.g., about 0.2 to 1%). PCT Publication No. WO 95/17210 describes exemplary emulsions.

The adjuvant designated QS21 is an immunologically active saponin fraction having adjuvant activity derived from the bark of the South American tree Quillaja Saponaria Molina, and the method of its production is disclosed in U.S. Pat. No. 5,057,540. Semi-synthetic and synthetic derivatives of Quillaj a Saponaria Molina saponins are also useful, such as those described in U.S. Pat. Nos. 5,977,081 and 6,080,725.

TLRs are a family of proteins homologous to the Drosophila Toll receptor, which recognize molecular patterns associated with pathogens and thus aid the body in distinguishing between self and non-self molecules. Substances common in viral pathogens are recognized by TLRs as pathogen-associated molecular patterns. For example, TLR-3 recognizes patterns in double-stranded RNA, TLR-4 recognizes patterns in lipopolysaccharides while TLR-7/8 recognize patterns containing adenosine in viral and bacterial RNA and DNA. When a TLR is triggered by such pattern recognition, a series of signaling events occurs that leads to inflammation and activation of innate and adaptive immune responses. A number of synthetic ligands containing the molecular patterns recognized by various TLRs are being developed as adjuvants and may be included in an immunogenic composition as described herein.

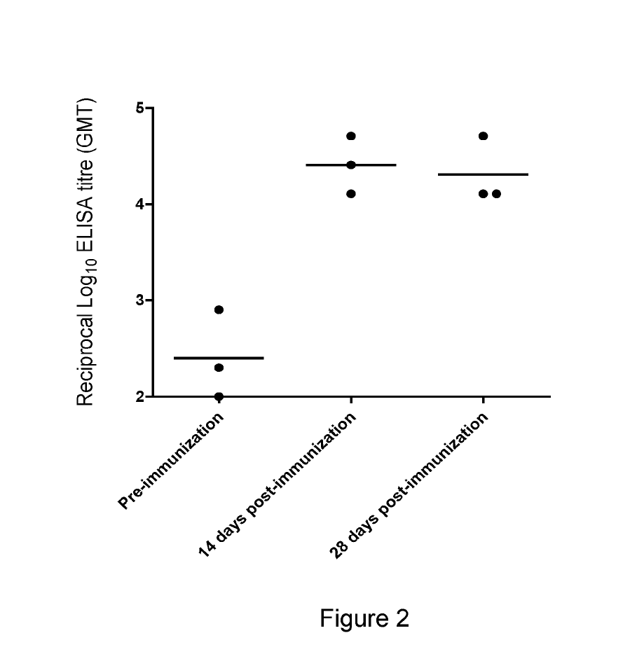

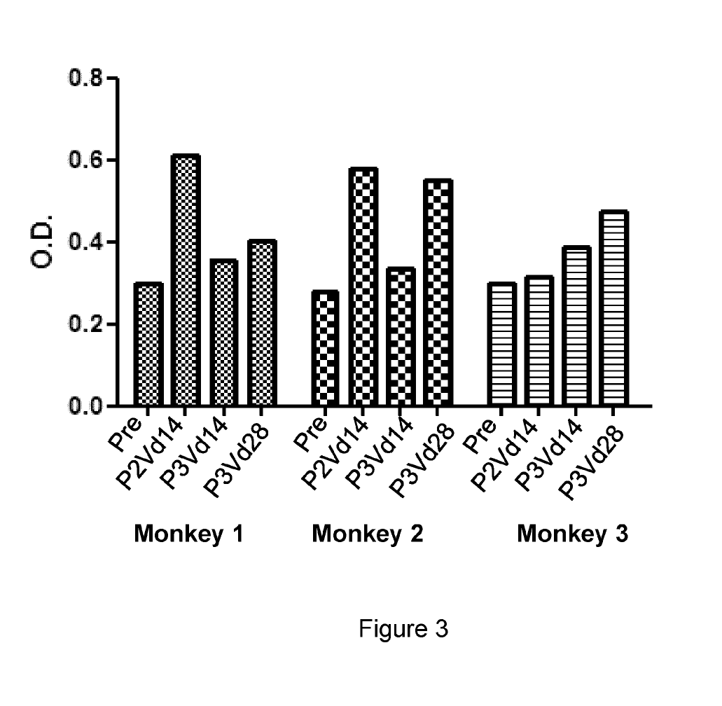

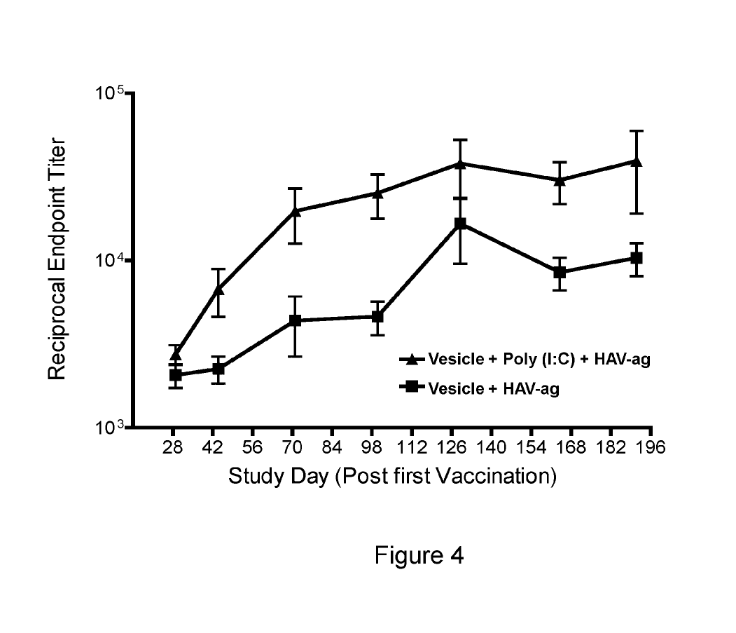

For example, polyriboinosinic:polyribocytidylic acid or poly(I:C), a synthetic double-stranded RNA, is an exemplary adjuvant that is an agonist for TLR-3. Examples 4 (FIGS. 2 and 3) and 5 (FIG. 4) were performed using immunogenic compositions which included this adjuvant. The data in these examples suggests that this TLR-3 agonist may be advantageously included in an immunogenic composition as described herein. Of note, the immunogenic composition used in Example 4 also included alum as an adjuvant since alum is present in the commercial HAVRIX which was used in preparing this composition. The data in this example therefore supports the utility of combining alum with a TLR-3 agonist such as poly(I:C) in an inventive composition. Another exemplary TLR-3 agonist is poly(IC:LC) which is a synthetic, double-stranded RNA poly(I:C) stabilized with poly-L-lysine carboxymethyl cellulose.

MPL™ and 3D-MPL™ are exemplary adjuvants that are agonists for TLR-4. These adjuvants contain monophosphoryl lipid A (MPL) and 3-deacyl monophosphoryl lipid A (3D-MPL), respectively in combination with trehalosedimycolate (TDM) and cell wall skeleton (CWS) in a 2% squalene/Tween™ 80 emulsion (e.g., see GB Patent No. 2122204). Imiquimod (1-isobutyl-1H-imidazo[4,5-c]quinolin-4-amine) is a small molecule agonist of TLR-7/8 which may be advantageously included in an immunogenic composition as described herein.

### IV. Dosage and Administration

The immunogenic compositions are useful for treating hepatitis A in humans including adults and children. In general however they may be used with any animal. In certain embodiments, the methods herein may be used for veterinary applications, e.g., canine and feline applications. If desired, the methods herein may also be used with farm animals, such as ovine, avian, bovine, porcine and equine breeds.

Immunogenic compositions described herein will generally be administered in such amounts and for such a time as is necessary or sufficient to induce an immune response. Dosing regimens may consist of a single dose or a plurality of doses over a period of time. The exact amount of viral antigen to be administered may vary from subject to subject and may depend on several factors. Thus, it will be appreciated that, in general, the precise dose used will be as determined by the prescribing physician and will depend not only on the weight of the subject and the route of administration, but also on the frequency of dosing, the age of the subject and the severity of the symptoms and/or the risk of infection. In certain embodiments, the dose of viral antigen in an immunogenic composition may range from about 0.1 μg to about 1 mg, e.g., from about 1 μg to about 1 mg, from about 10 μg to about 1 mg, from about 50 μg to about 1 mg, from about 100 μg to about 750 μg, from about 200 μg to about 500 μg, from about 0.1 μg to about 0.5 μg, from about 0.1 μg to about 1 μg, from 0.1 μg to about 5 μg, from about 0.1 μg to about 10 μg, from about 0.1 μg to about 100 μg, from 1 μg to about 5 μg, from about 1 μg to about 10 μg, from about 1 μg to about 50 μg, from about 10 μg to about 100 μg, etc. As is well known in the art, the standard used to express viral antigen activity for HAVRIX is derived from an ELISA, with results reported in, “ELISA Units”, or EL.U. One ELISA Unit is equivalent to about 0.42 ng of HAV antigen. It is to be understood that each of the aforementioned ranges could be converted into an alternative EL.U range by dividing the mass by 0.42 ng. In certain embodiments, the dose of viral antigen in an immunogenic composition may range from about 180 EL.U. to about 8640 EL.U., e.g., from about 180 EL.U. to about 8640 EL.U, from about 360 EL.U. to about 8640 EL.U, from about 720 EL.U. to about 8640 EL.U, from about 1440 EL.U. to about 8640 EL.U, from about 2880 EL.U. to about 8640 EL.U, from about 5760 EL.U. to about 8640 EL.U, etc. Lower doses of viral antigen may be sufficient when using sublingual or buccal administration, or in the presence of adjuvant. Higher doses may be more useful when given orally, especially in the absence of adjuvants.

In general, the compositions may be administered to a subject by any route. In particular, the results in the Examples demonstrate that the immunogenic compositions described herein can induce a protective response even when administered orally. It will be appreciated that the oral route is particularly desirable in light of the advantages of oral delivery over any form of injection (i.e., compliance, mass distribution, etc.). It will also be appreciated that the results are unexpected in light of the fact that most vaccines and all known hepatitis A vaccines have so far been administered parenterally.

Thus, in certain embodiments, the compositions may be administered orally (including buccally, sublingually and by gastric lavage or other artificial feeding means). Such oral delivery may be accomplished using solid or liquid compositions, for example in the form of tablets, capsules, multi-particulates, gels, films, ovules, elixirs, solutions, suspensions, etc. In certain embodiments, when using a liquid composition, the composition may be administered in conjunction with a basic composition (e.g., a bicarbonate solution) in order to neutralize the stomach pH. In certain embodiments, the basic composition may be administered before and/or after the immunogenic composition. In certain embodiments, the basic composition may be combined with the immunogenic composition prior to administration or taken at the same time as the immunogenic composition.

While oral delivery is of particular interest, it will be appreciated that in certain embodiments, an immunogenic composition may also be formulated for delivery parenterally, e.g., by injection. In such embodiments, administration may be, for example, intravenous, intramuscular, intradermal, or subcutaneous, or via by infusion or needleless injection techniques. For such parenteral administration, the immunogenic compositions may be prepared and maintained in conventional lyophylized compositions and reconstituted prior to administration with a pharmaceutically acceptable saline solution, such as a 0.9% saline solution. The pH of the injectable composition can be adjusted, as is known in the art, with a pharmaceutically acceptable acid, such as methanesulfonic acid. Other acceptable vehicles and solvents that may be employed include Ringer's solution and U.S.P. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

The immunogenic compositions can also be administered intranasally or by inhalation and are conveniently delivered in the form of a dry powder inhaler or an aerosol spray presentation from a pressurized container, pump, spray, atomiser or nebuliser, with or without the use of a suitable propellant, e.g., dichlorodifluoromethane, trichlorofluoromethane, dichlorotetrafluoroethane, a hydrofluoroalkane, carbon dioxide or other suitable gas. In the case of a pressurized aerosol, the dosage unit may be determined by providing a valve to deliver a metered amount. The pressurized container, pump, spray, atomiser or nebuliser may contain a solution or suspension of the antibody, e.g., using a mixture of ethanol and the propellant as the solvent, which may additionally contain a lubricant, e.g., sorbitantrioleate. Capsules and cartridges (made, for example, from gelatin) for use in an inhaler or insufflator may be formulated to contain a powder mix of the immunogenic composition and a suitable powder base such as lactose or starch.

Compositions for rectal administration are preferably suppositories which can be prepared by mixing the immunogenic composition with suitable non-irritating excipients or carriers such as cocoa butter, polyethylene glycol or a suppository wax which are solid at ambient temperature but liquid at body temperature and therefore melt in the rectal vault and release the antibodies. Retention enemas and rectal catheters can also be used as is known in the art. Viscosity-enhancing carriers such as hydroxypropyl cellulose are also certain carriers of the invention for rectal administration since they facilitate retention of the composition within the rectum. Generally, the volume of carrier that is added to the composition is selected in order to maximize retention of the composition. In particular, the volume should not be so large as to jeopardize retention of the administered composition in the rectal vault.

### EXAMPLES

The following examples describe some exemplary modes of making and practicing certain compositions that are described herein. It should be understood that these examples are for illustrative purposes only and are not meant to limit the scope of the compositions and methods described herein.

### Example 1

### Vesicle Preparation

This example describes the preparation of exemplary vesicles using the chloroform melt method that was used in Example 4. A 5:4:1 molar ratio of lipids:monopalmitoyl-glycerol (MPG), cholesterol (CHO) and dicetyl phosphate (DCP) was placed in a round bottom 250 ml flask, ensuring none of the powder stuck to the side of the glass flask. The lipids were then dissolved in chloroform and the mixture dried on a rotary evaporator. Evaporation was allowed to continue until the solvent was completely removed and a dry lipid layer coated the flask which was then transferred to a desiccator and maintained under vacuum overnight.

The dried mixture was resuspended in 25 mM sodium bicarbonate solution pH 7.6 at 50° C. and 100 mM sodium deoxycholate in 25 mM sodium bicarbonate solution pH 9.7. If included, adjuvant and/or antigen solution was added at this step. After cooling to 37° C., the solution was mixed for 8 hours, dispensed into vials, frozen at −70° C. and lyophilized.

Another method, the three step melt method was used in the preparation of exemplary vesicles that were used in Example 5. In this method the use of chloroform was eliminated. Briefly, the 5:4:1 molar ratio of MPG, CHO and DCP lipid mixture described above was added in powder form and melted at 120° C. with mixing. An emulsion was formed by homogenizing at 50° C. with 25 mM sodium bicarbonate solution pH 7.6, with further homogenization after the subsequent addition of 100 mM sodium deoxycholate in 25 mM sodium bicarbonate solution pH 9.7. If included, adjuvant and/or antigen solution was added at this step. After cooling to 30° C., the solution was mixed for 2 hours, dispensed into vials, frozen at −70° C. and lyophilized.

For both methods of vesicle preparation described above, poly (I:C), poly (IC:LC) and/or antigen can be introduced during vesicle preparation prior to the last mixing step and lyophilization. Alternatively, they can be added and incorporated during reconstitution of the dried vesicles after lyophilization by mixing with reconstitution buffer, e.g., sodium bicarbonate reconstitution buffer, prior to dosing.

As discussed in the following examples, the resulting vesicles were evaluated for size using dynamic light scattering and for entrapment efficiency using a Ninhydrin assay or a non competitive sandwich ELISA. Loaded vesicles were then adjusted in volume to produce a composition with the desired quantity of hepatitis A antigen.

### Example 2

### Physiochemical Characterization of Vesicles

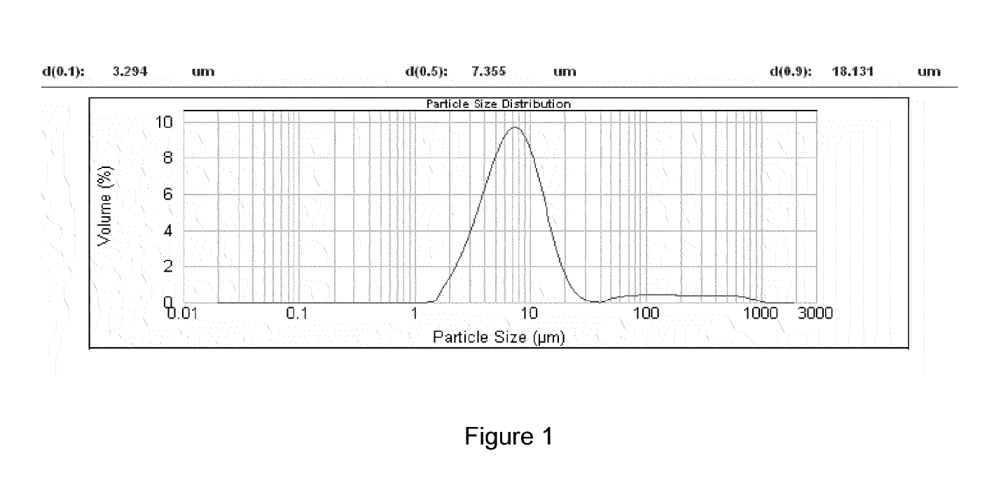

This example describes how the dimensions of the vesicles were determined using dynamic light scattering. Visual inspection indicated that the vesicle formulation (with entrapped poly (I:C) and hepatitis A antigen) was a milky white solution. We determined particle size and size distribution using a Malvern Instrument MASTERSIZER 2000 (Hydro 2000S) using triplicate readings and a 2 min equilibration time. Approximately 250 μl of vesicle sample was added to 25 mM bicarbonate buffer pH 9.7 in a sample well. Statistical analysis was performed using Minitab v14 with a 2-sample t-test at the 95% confidence level. The results obtained from the MASTERSIZER analysis are shown in FIG. 1.

### Example 3

### Analysis of Hepatitis A Antigen Entrapment

This example describes how the level of hepatitis A antigen entrapment was determined using a Ninhydrin assay. The Ninhydrin assay is a colorimetric method of determining the concentration of a protein in a sample. Substances containing amino groups react with the Ninhydrin reagent to yield a blue-purple complex. The hepatitis A antigens were hydrolyzed from the lipid vesicle, neutralized, mixed with ninhydrin reagent and then incubated at 110° C. The solution was then allowed to cool and its absorbance was measured at 595 nm. There is a linear relationship between absorbance at this wavelength and the amount of protein present in the original sample. Alternatively the level of hepatitis A antigen entrapment was determined using a non competitive (sandwich) ELISA. Vesicle samples were centrifuged to obtain a supernatant and pellet. The pellet was extracted with 0.5% Triton X-100 and sonicated. The level of hepatitis A antigen entrapped was determined by sandwich ELISA which measures the amount of antigen in the sample between two layers of antibodies (i.e., capture and detection antibodies).

### Example 4

### Hepatitis A Immunization of Monkeys

This Example describes in vivo testing of certain immunogenic compositions in monkeys. The objective of this study was to determine the immunogenicity of a preparation of commercially available hepatitis A antigen (HAVRIX) when administered orally in a freeze-dried vesicle preparation to Rhesus macaques. HAVRIX is a formaldehyde inactivated HM175 strain of hepatitis A virus supplied as a suspension in phosphate buffer. It is commercially available from GlaxoSmithKline and the usual adult human dose is 1440 U adsorbed to 0.5 mg aluminum as aluminum hydroxide in a 0.5 ml IM dose. In this example vesicles were prepared by the chloroform melt method of Example 1 and hepatitis A antigen (HAVRIX) and poly (I:C) adjuvant were added prior to mixing such that antigen and adjuvant were incorporated during vesicle preparation. Rhesus macaques (n=3) were vaccinated three times by gastric gavage with the vesicles on days 0, 14, and 28 (equivalent of 4320 U/dose):

| Species/ | | | Gender and | Dose |
|---|---|---|---|---|
| Strain | Route | Duration | No. per Group | Frequency |
| Rhesus | Oral | 56 days | 2♂ 1♀/group | Days 0, 14 and 28 |
| macaques | | 42 days | | |

Serum samples were subsequently collected to assess hepatitis A-specific IgG titers induced by vaccination. Serum samples collected 14 days and 28 days after the last immunization were tested by ELISA against inactivated hepatitis A viral antigen. Data was plotted as geometric mean titre from end point dilutions of monkey sera. As shown in FIG. 2, oral vaccination of monkeys with HAVRIX and poly (I:C) in a vesicle formulation, showed an induced systemic (serum) IgG response against the hepatitis A antigen 14 and 28 days after the third vaccination. The response was comparable to or greater than that seen in monkeys immunized by IM injection (data not shown).

Nasal wash samples were also collected to assess hepatitis A-specific mucosal IgA responses. Nasal wash samples collected 14 days after the second vaccination (day 28), and 14 and 28 days after the third vaccination (day 42 and day 56, respectively) were tested by ELISA against inactivated hepatitis A viral antigen. As shown in FIG. 3, oral vaccination of monkeys induced mucosal (nasal wash) IgA responses against the hepatitis A antigen throughout the time period.

A number of researchers have demonstrated that currently licensed hepatitis A vaccines given by intramuscular (IM) injection induce neutralizing IgG antibodies. As discussed above, we have shown that orally administered immunogenic hepatitis A compositions are capable of inducing IgG antibodies systemically (serum samples) and IgA antibodies mucosally (nasal wash samples). Since hepatitis A infection occurs via mucosal surfaces, an IgA response (the hallmark of a mucosal immune response) may be more efficacious than a systemic IgG response. We would only expect systemic IgG responses if the immunogenic hepatitis A compositions were to be administered by standard parenteral routes (e.g., by IM injection).

### Example 5

### Hepatitis A Immunization of Mice

This Example describes in vivo testing of certain immunogenic compositions in mice. The objective of this study was to determine 1) the durability of immunogenicity induced by oral vaccination and 2) the potential value of an adjuvant, using a commercially available hepatitis A antigen (HAV-ag) from Meridian Life Sciences when administered orally in a freeze-dried vesicle preparation to BALB/c mice. Meridian HAV-ag is a hepatitis A virus strain HM175, which was grown in FRhK-4 cells, partially purified by gradient centrifugation, and inactivated by treatment with formalin. It is similar to the antigen used in Example 4 (HAVRIX) in that it is the same hepatitis A strain and is formalin-inactivated; however unlike HAVRIX, Meridian HAV-ag is grown in a different cell line, and does not contain aluminum hydroxide. In this example vesicles were prepared by either the chloroform or three step melt methods and hepatitis A antigen (Meridian HAV-ag) and poly(I:C) adjuvant were either added prior to mixing such that antigen and adjuvant were incorporated during vesicle preparation and prior to lyophilization or they were added during reconstitution of the lyophilized vesicle preparations. BALB/c mice were sequentially gavaged with the variously formulated vesicle/HAV-ag compositions. Doses containing 2 μg antigen were administered orally on days 0, 3, 14 and 17:

| Species/ | | | Gender and | Dose |
|---|---|---|---|---|
| Strain | Route | Duration | No. per Group | Frequency |
| BALB/c | Oral | 56 days | 8 ♀/group | Days 0, 3, 14 and 17 |
| mice | | | | |

Serum samples were subsequently collected to assess hepatitis A-specific IgG titers induced by vaccination. Serum samples were collected prior to study start and on days 10, 29, 44, 71, 100, 129, 164 and 191 of the study and were tested by ELISA against inactivated hepatitis A viral antigen. Data was plotted as geometirc mean titre from end point dilutions of mouse sera. As shown in FIG. 4 mice in all groups showed the presence of serum IgG to hepatitis A 29 days after first oral vaccination (12 days after the fourth dose). There was a sustained IgG titer in all groups for up to 191 days post first dose (6 months after the fourth dose).

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims. The entire contents of any reference that is referred to herein are hereby incorporated by reference.

1. An immunogenic composition comprising:
an inactivated or attenuated hepatitis A virus; and
a vesicle which comprises a non-ionic surfactant and a transport enhancer which facilitates the transport of lipid-like molecules across mucosal membranes

2. The composition of claim 1, where the composition comprises an inactivated hepatitis A virus.

3. The composition of claim 1, where the composition comprises an attenuated hepatitis A virus.

4. The composition of claim 1, where the non-ionic surfactant is a glycerol ester.

5. The composition of claim 1, where the non-ionic surfactant is a glycol or glycerol ether.

6. The composition of claim 1, where the transport enhancer is a cholesterol derivative in which the C₂₋₃ carbon atom of the side chain carries a carboxylic acid.

7. The composition of claim 1, where the transport enhancer is cholic acid, chenodeoxycholic acid or a salt thereof.

8. The composition of claim 1, where the transport enhancer is glycocholic acid, taurocholic acid, deoxycholic acid, ursodeoxycholic acid, or a salt thereof.

9. The composition of claim 1, where the transport enhancer is an acyloxylated amino acid or a salt thereof.

10. The composition of claim 1, where the transport enhancer is an acylcarnitine containing a C₆₋₂₀alkanoyl or alkenoyl moiety or a salt thereof.

11. The composition of claim 1, where the vesicle further comprises an ionic amphiphile.

12. The composition of claim 11, where the ionic amphiphile is an alkanoic acid or an alkenoic acid.

13. The composition of claim 11, where the ionic amphiphile is a phosphate.

14. The composition of claim 11, where the ionic amphiphile is dicetylphospate, phosphatidic acid or phosphatidyl serine.

15. The composition of claim 11, where the ionic amphiphile is a sulphate monoester.

16. The composition of claim 11, where the ionic amphiphile is cetylsulphate.

17. The composition of claim 1, where the vesicle further comprises a steroid.

18. The composition of claim 17, where the steroid is cholesterol.

19. The composition of claim 1, where the vesicle has a diameter in the range of about 150 nm to about 10 μm.

20. The composition of claim 1, where the vesicle has a diameter in the range of about 800 nm to about 1.5 μm.

21. The composition of claim 1, where the vesicle has a diameter which is greater than 10 μm.

22. The composition of claim 1, where the inactivated or attenuated hepatitis A virus is encapsulated within an aqueous core of the vesicle.

23. The composition of claim 1, where the composition further comprises an adjuvant.

24. The composition of claim 23, where the adjuvant is a TLR-3 agonist.

25. The composition of claim 24, where the TLR-3 agonist is polyriboinosinic:polyribocytidylic acid.

26. The composition of claim 24, where the TLR-3 agonist is polyriboinosinic:polyribocytidylic acid stabilized with poly-L-lysine carboxymethyl cellulose.

27. The composition of claim 24, where the composition further comprises alum.

28. The composition of claim 25, where the composition further comprises alum.

29. The composition of claim 26, where the composition further comprises alum.

30. A method of treating an individual suffering from, or at risk for, hepatitis A, the method comprising administering to the individual a therapeutically effective amount of an immunogenic composition comprising:
an inactivated or attenuated hepatitis A virus; and
a vesicle which comprises a non-ionic surfactant and a transport enhancer which facilitates the transport of lipid-like molecules across mucosal membranes.

31. The method of claim 30, where the composition is administered orally.

32. The method of claim 31, where the composition comprises between about 0.1 μg and about 1 mg of inactivated or attenuated hepatitis A virus.

33-63. (canceled)
